(19) United States
(12) Patent Application Publication
(10) Pub. No.: US 2025/0235692 A1
(43) Pub. Date: Jul. 24, 2025

(54) BIOLOGICAL SEALING FOR NEURAL INTERFACES

(71) Applicant: PRECISION NEUROSCIENCE CORPORATION, New York, NY (US)

(72) Inventors: Mark HETTICK, New York, NY (US); Stephanie RIDER, New York, NY (US); Demetrios PAPAGEORGIOU, New York, NY (US); Manuel MONGE, New York, NY (US); Benjamin RAPOPORT, New York, NY (US)

(73) Assignee: PRECISION NEUROSCIENCE CORPORATION, New York, NY (US)

(21) Appl. No.: 19/032,815

(22) Filed: Jan. 21, 2025

Related U.S. Application Data
(60) Provisional application No. 63/622,226, filed on Jan. 18, 2024.

(51) Int. Cl. A61N 1/05

(52) U.S. Cl. CPC A61N 1/0539

(57) ABSTRACT

Systems and methods for hermetic sealing a neural interface. The neural interface can include a substrate, an electronics subassembly disposed on the substrate, an electrode array coupled to the substrate defining a first interface, wherein the electrode array comprises a plurality of electrodes disposed at a distal end thereof, a lead wire coupled to the substrate, and an encapsulation layer covering the electronics, the first interface, and the second interface. The encapsulation layer comprises a low vapor permeability material and is configured to hermetically seal the electronics, the first interface, and the second interface from a biological environment.

### CROSS-REFERENCE TO RELATED APPLICATIONS

The present application claims priority to U.S. Provisional Patent Application No. 63/622,226, titled BIOLOGICAL SEALING FOR NEURAL INTERFACES, filed Jan. 18, 2024, which is hereby incorporated by reference herein in its entirety.

### BACKGROUND

Brain-computer interfaces have shown promise as systems for restoring, replacing, and augmenting lost or impaired neurological function in a variety of contexts, including paralysis from stroke and spinal cord injury, blindness, and some forms of cognitive impairment. Multiple innovations over the past several decades have contributed to the potential of these neural interfaces, including advances in the areas of applied neuroscience and multichannel electrophysiology, mathematical and computational approaches to neural decoding, power-efficient custom electronics and the development of application-specific integrated circuits, as well as materials science and device packaging. Nevertheless, the practical impact of such systems remains limited, with only a small number of patients worldwide having received highly customized interfaces through clinical trials.

High bandwidth brain-computer interfaces are being developed to enable the bidirectional communication between the nervous system and external computer systems in order to assist, augment, or replace neurological function lost to disease or injury. A necessary capability of any brain-computer interface is the ability to accurately decode electrophysiologic signals recorded from individual neurons, or populations of neurons, and correlate such activity with one or more sensory stimuli or intended motor response. For example, such a system can record activity from the primary motor cortex in an animal or a paralyzed human patient and attempt to predict the actual or intended movement in a specific body part; or the system can record activity from the visual cortex and attempt to predict both the location and nature of the stimuli present in the patient's visual field.

Furthermore, brain-penetrating microelectrode arrays have facilitated high-spatial-resolution recordings for brain-computer interfaces, but at the cost of invasiveness and tissue damage that scale with the number of implanted electrodes. In some applications, softer electrodes have been used in brain-penetrating microelectrode arrays; however, it is not yet clear whether such approaches offer a substantial improvement as compared to conventional brain-penetrating electrodes. For this reason, non-penetrating cortical surface microelectrodes represent a potentially attractive alternative and form the basis of the system described here. In practice, electrocorticography (ECoG) has already facilitated capture of high-quality signals for effective use in brain-computer interfaces in several applications, including motor and speech neural prostheses. Higher-spatial-resolution micro-electrocorticography (u ECoG) therefore represents a promising combination of minimal invasiveness and improved signal quality.

Because neural interfaces are at least partially implanted into the brain, it is necessary to hermetically seal any portions of the neural interfaces that can come in contact with biological structures and/or biological fluids in order to (i) prevent biological fluids and biological material from damaging the electronics and (ii) preventing the electronics from interacting with the subject's body. In particular, the leakage from electronics into the body directly can be harmful to the patient.

### SUMMARY

The present disclosure is directed to system and methods for hermetically sealing neural interfaces from biological environments, thereby preventing damage to said components or otherwise preventing the materials of said components from interacting with the biological environment.

In one embodiment, there is provided a neural interface for subdural implantation, the neural interface comprising: a substrate; an electronics subassembly disposed on the substrate; an electrode array coupled to the substrate defining a first interface, wherein the electrode array comprises a plurality of electrodes disposed at a distal end thereof; a lead wire coupled to the substrate defining a second interface; and an encapsulation layer covering the electronics subassembly, the first interface, and the second interface, wherein the encapsulation layer comprises a low vapor permeability material, and wherein the encapsulation layer hermetically seals the electronics subassembly, the first interface, and the second interface from a biological environment.

In some embodiments, the substrate is flexible.

In some embodiments, the electronics subassembly comprise an application-specific integrated circuit.

In some embodiments, the encapsulation layer comprises a multilayer stack of materials.

In some embodiments, the low vapor permeability material comprises a glass, a plastic, a ceramic, or a combination thereof.

In some embodiments, the electronics subassembly comprises at least one of a redistribution layer, a passive layer, or an application-specific integrated circuit.

In some embodiments, the encapsulation layer comprises a thin film encapsulation layer.

In some embodiments, the encapsulation layer comprises at least one mechanical feature configured to interface with external systems.

In some embodiments, the at least one mechanical feature is at least one of a loop, mounting hole, a matching shape, or a mount.

In some embodiments, the neural interface further comprises an internal encapsulation assembly configured to cover the electronics subassembly, wherein the encapsulation layer covers the internal encapsulation assembly.

In some embodiments, the internal encapsulation assembly comprises glass.

In some embodiments, the internal encapsulation assembly defines a sealed inert atmosphere interior cavity.

In some embodiments, the electrode array comprises 1,000 or more electrodes.

In one embodiment, there is provided a method of fabricating a neural interface for subdural implantation, the method comprising: attaching an electrode array to a first portion of a substrate defining a first interface, wherein the electrode array comprises a plurality of electrodes; attaching a lead wire to a second portion of the substrate defining a second interface; attaching an electronics subassembly to the substrate; and forming an encapsulation layer covering the first interface, the second interface, and the electronics subassembly, wherein the encapsulation layer comprises a low vapor permeability material, wherein the encapsulation layer hermetically seals the electronics subassembly, the first interface, and the second interface from a biological environment.

In some embodiments, the encapsulation layer is formed by welding or sealing the encapsulation layer to the substrate.

Further features of the present disclosure, as well as the structure and operation of various embodiments, are described in detail below with reference to the accompanying drawings. It is noted that the present disclosure is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE FIGURES

The accompanying drawings, which are incorporated herein and form part of the specification, illustrate the present disclosure and, together with the description, further serve to explain the principles of the present disclosure and to enable a person skilled in the relevant art(s) to make and use embodiments described herein.

FIG. 1 illustrates an illustrative system including a neural device, in accordance with an embodiment of the present disclosure.

FIG. 2 depicts a diagram of a neural device, in accordance with an embodiment of the present disclosure.

FIG. 3 depicts a diagram of a thin-film, microelectrode array neural device and implantation method, in accordance with an embodiment of the present disclosure.

FIG. 4 illustrates a sectional view of a first embodiment of a hermetically sealed neural interface, in accordance with an embodiment of the present disclosure.

FIG. 5 illustrates a sectional view of a second embodiment of a hermetically sealed neural interface, in accordance with an embodiment of the present disclosure.

FIG. 6 illustrates a sectional view of a third embodiment of a hermetically sealed neural interface, in accordance with an embodiment of the present disclosure.

FIG. 7 illustrates a perspective sectional phantom view of an embodiment of a hermetically sealed neural interface, in accordance with an embodiment of the present disclosure.

FIG. 8 illustrates a perspective view of an embodiment of a hermetically sealed neural interface, in accordance with an embodiment of the present disclosure.

FIG. 9 is a graph delineating hermetic and non-hermetic materials by Young's Modulus.

### DETAILED DESCRIPTION

The present disclosure is generally directed to systems and methods for hermetically encapsulating neural interfaces to protect electronics from being exposed to biological fluids and/or biological material. Neural interfaces are at least partially implanted into the brain and any portion of the neural interface can come into contact with biological structures and/or biological fluids in the body. By hermetically sealing those portions of the neural interface, damage to the electronics of the neural interface and leakage into the body from the damaged electronics can be prevented. Additionally, the hermetic seal can prevent the electronics from being damaged such that the neural interface does not suffer a loss in functionality.

### Neural Interfaces

Progress toward the development of brain-computer interfaces has signaled the potential to restore, replace, and augment lost or impaired neurological function of humans in a variety of disease states. Existing approaches to developing high-bandwidth brain-computer interfaces rely on invasive surgical procedures or brain-penetrating electrodes, which limit addressable applications of the technology and the number of eligible patients.

As noted above, typical neural devices include electrode arrays that penetrate a user's brain in order to sense and/or stimulate the brain. However, the present disclosure is directed to the use of non-penetrating neural devices, i.e., neural devices having electrode arrays that do not penetrate the cortical surface. Such non-penetrating neural devices are minimally invasive and minimize the amount of impact on the user's cortical tissue. Neural devices can sense and record brain activity, receive instructions for stimulating the user's brain, and otherwise interact with a user's brain as generally described herein.

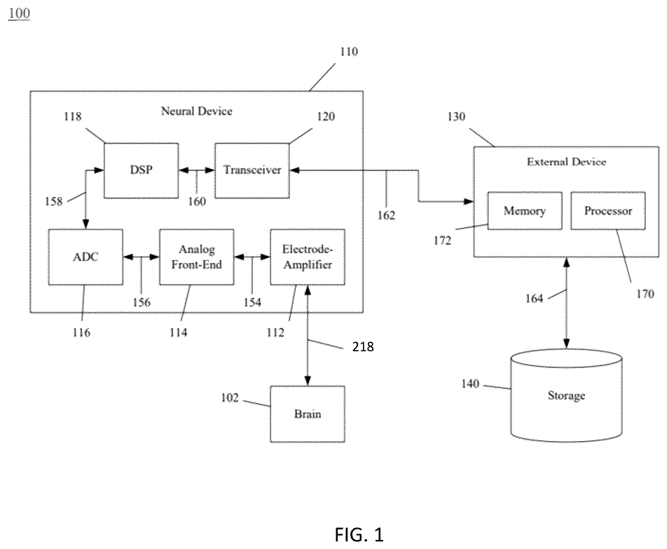

Referring now to FIG. 1, there is shown a diagram of an illustrative system 100 including a neural device 110 that is communicatively coupled to an external device 130. The external device 130 can include any device to which the neural device 110 can be communicatively coupled, such as a computer system or mobile device (e.g., a tablet, a smartphone, a laptop, a desktop, a secure server, a smartwatch, a head-mounted virtual reality device, a head-mounted augmented reality device, or a smart inductive charger device). The external device 130 can include a processor 140 and a memory 142. In some embodiments, the computer system or mobile device can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure). The external device 130 can include a processor 170 and a memory 172. In some embodiments, the external device 130 can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure).

In some embodiments, the electrode array 218 of the neural device 110 can have electrodes that are sufficiently small and spaced at sufficiently small distances in order to define a high-density electrode array 218 that can, accordingly, capture high resolution electrocortical data. Such high-resolution data can be used to resolve electrographic features that can otherwise not be identified using lower resolution electrode arrays. In some embodiments, the electrodes of the electrode array 218 can be from about 10 μm to about 500 μm in width. In one illustrative embodiment, the electrodes of the electrode array 218 can be about 50 μm in width. In some embodiments, the electrodes of the electrode array 218 can be spaced by about 200 μm (i.e., 0.2 mm) to about 3,000 μm (i.e., 3 mm). In illustrative one embodiment, adjacent electrodes of the electrode array 218 can be spaced by about 400 μm. The electrode array 218 can be constructed from a variety of different materials and utilizing different techniques to facilitate longevity, such as are disclosed in U.S. patent application Ser. No. 18/829,780, titled ELECTRODE ARRAY LONGEVITY, filed Sep. 10, 2024, which is hereby incorporated by reference herein in its entirety.

The neural device 110 can include a range of electrical or electronic components. In the illustrated embodiment, the neural device 110 includes an electrode-amplifier stage 112, an analog front-end stage 114, an analog-to-digital converter (ADC) stage 116, a digital signal processing (DSP) stage 118, and a transceiver stage 120 that are communicatively coupled together. The electrode-amplifier stage 112 can include an electrode array, such as is described below, that is able to physically interface with the brain of the subject 102 in order to sense brain signals and/or apply electrical signals thereto. The analog front-end stage 114 can be configured to amplify signals that are sensed from or applied to the subject 102, perform conditioning of the sensed or applied analog signals, perform analog filtering, and so on. The front-end stage 114 can include, for example, one or more application-specific integrated circuits (ASICs) or other electronics. The ADC stage 116 can be configured to convert received analog signals to digital signals and/or convert received digital signals to an analog signal to be processed via the analog front-end stage 114 and then applied via the electrode-amplifier stage 112. The DSP stage 118 can be configured to perform various DSP techniques, including multiplexing of digital signals received via the electrode-amplifier stage 112 and/or from the external device 130. For example, the DSP stage 118 can be configured to convert instructions from the external device 130 to a corresponding digital signal. The transceiver stage 120 can be configured to transfer data from the neural device 110 to the external device 130 located outside of the body of the subject 102.

The analog front-end stage 114 can be configured to amplify signals that are sensed from or applied to the brain 102, perform conditioning of the sensed or applied analog signals, perform analog filtering, and so on. The front-end stage 114 can include, for example, one or more application-specific integrated circuits (ASICs) or other electronics, such as the ADC stage 116. The ADC stage 116 can be configured to convert received analog signals to digital signals.

The DSP stage 118 can be configured to perform various DSP techniques, including multiplexing of digital signals and/or reducing the data rate by performing a feature extraction of or data compression the digitized signals received via the electrode-amplifier stage 112 and/or from the external device 130. For example, the DSP stage 118 can be configured to convert instructions from the external device 130 to a corresponding digital signal. The transceiver stage 120 can be configured to transfer data from the neural device 110 to the external device 130 located outside of the body of the user.

In various embodiments, the stages of the neural device 110 can provide unidirectional or bidirectional communications in half-or full-duplex mode (as indicated in FIG. 1) by and between the neural device 110 and the external device 130. In various embodiments, one or more of the stages can operate in a serial or parallel manner with other stages of the system 100. It can further be noted that the depicted architecture for the system 100 is simply intended for illustrative purposes and that the system 100 can be arranged differently (i.e., components or stages can be connected in different manners) or include additional components or stages.

In some embodiments, the electrode array 218 can include non-penetrating microelectrodes. In one embodiment, the electrode array 218 can include 500 or more electrodes. In another embodiment, the electrode array 218 can include 1,000 or more electrodes. In one illustrative embodiment, the electrode array 218 can include 1,024 electrodes.

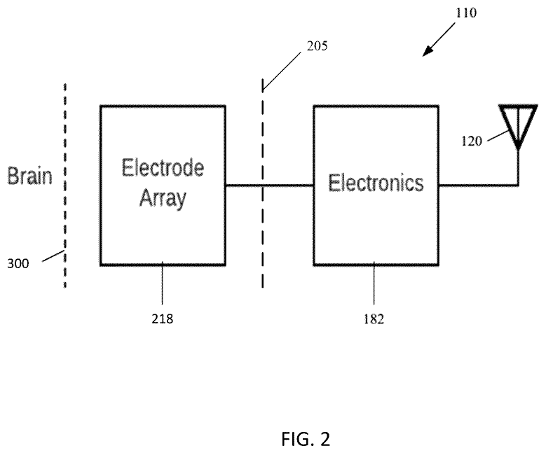

In some embodiments, the neural device 110 described above can include a brain implant, such as is shown in FIG. 2. The neural device 110 can be a biomedical device configured to study, investigate, diagnose, treat, and/or augment brain activity. In some embodiments, the neural device 110 can be a subdural neural device, i.c., a neural device implanted between the dura 205 (i.e., the membrane surrounding the brain) and the cortical surface of the brain 300. In some embodiments, the neural device 110 can be positioned beneath the dura mater 205 or between the dura mater 205 and the arachnoid membrane. In some embodiments, the neural device 110 can be positioned in the subdural space, on the cortical surface of the brain 300. The neural device 110 can be inserted through an incision in the scalp 302 and across the dura 205. The neural device 110 can include an electrode array 218 (which can be a component of or coupled to the electrode-amplifier stage 112 described above) that is configured to record and/or stimulate an area of the brain 300. The electrode array 218 can be connected to an electronics hub (which can include one or more of the electrode-amplifier stage 112, analog front-end stage 114, ADC stage 116, and DSP stage 118) that is configured to transmit via wireless or wired transceiver 120 to the external device 130 (in some cases, referred to as a “receiver”).

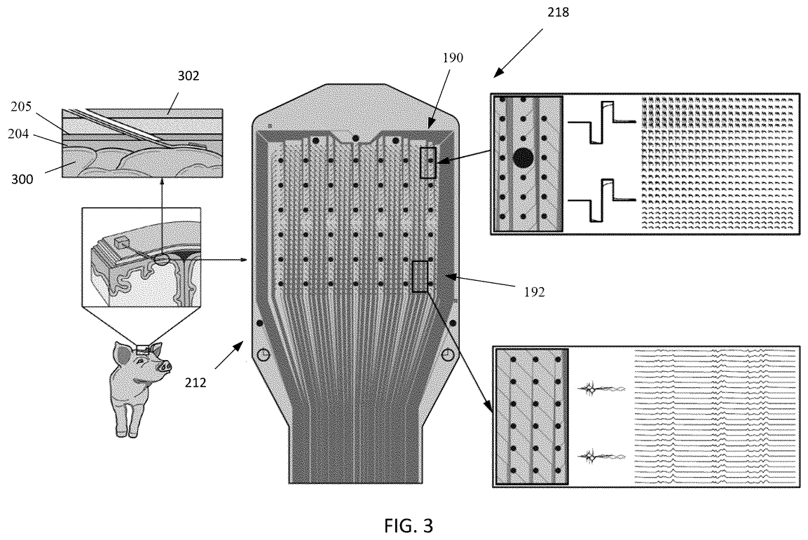

Referring now to FIG. 3, there is shown a diagram of an illustrative embodiment of a neural device 110. In this embodiment, the neural device 110 comprises an electrode array 218 comprising nonpenetrating microelectrodes. As generally described above, the neural device 110 is configured for minimally invasive subdural implantation using a cranial micro-slit technique, i.e., is inserted into the subdural space 204 between the dura and the surface of the subject's brain 300. In some embodiments, the neural device 110 is inserted into the subdural space 204 between the dura and the surface of the brain 300. Further, the microelectrodes of the electrode array 218 can be arranged in a variety of different configurations and can vary in size. In this particular example, the electrode array 218 includes a first group 190 of electrodes (e.g., 200 μm microelectrodes) and a second group 192 of electrodes (e.g., 20 μm microelectrodes). Further, example stimulation waveforms in connection with the first group 190 of electrodes and the resulting post-stimulus activity recorded over the entire array is depicted for illustrative purposes. Still further, example traces from recorded neural activity recorded by the second group 192 of electrodes are likewise illustrated. In this example, the electrode array 218 provides multichannel data that can be used in a variety of electrophysiologic paradigms to perform neural recording of both spontaneous and stimulus-evoked neural activity as well as decoding and focal stimulation of neural activity across a variety of functional brain regions.

Additional information regarding brain-computer interfaces described herein can be found in Ho et al., The Layer 7 Cortical Interface: A Scalable and Minimally Invasive Brain Computer Interface Platform, bioRxiv 2022.01.02.474656; doi: https://doi.org/10.1101/2022.01.02.474656, which is hereby incorporated by reference herein in its entirety.

### Biological Sealing for Neural Interfaces

Cardiac pacemakers, deep brain stimulators, spinal cord stimulators, and other types of implanted medical devices utilize a variety of forms of biological sealing to protect their electronic components. Unlike these conventional implanted medical devices, the neural interfaces 100 described herein can be disposed on flexible substrates to allow the electrode array to be surgically inserted using the minimally invasive surgical techniques described in, for example, U.S. patent application Ser. No. 18/434,008, titled MINIMALLY INVASIVE INSERTION SYSTEM FOR NEURAL INTERFACES, filed Feb. 6, 2024, which is hereby incorporated by reference herein in its entirety. The flexibility requirements for the neural interfaces 100 introduce two key issues. First, it would be beneficial for the housing or materials used to hermetically seal the electronics and other components of the neural interfaces 100 to likewise have sufficient flexibility to allow the neural interfaces 100 to be implanted using the aforementioned techniques. Second, at least two components of the neural interface 100 are exposed: (i) the distal or sensing portion of the electrode array and (ii) the transcutaneous lead wire that connects the implanted portion of the neural interface 100 to the extracorporeal portion (e.g., the power supply). This creates at least two locations where these externally extending components penetrate the housing. Accordingly, it would be beneficial for these penetration points to likewise be hermetically sealed in order to prevent fluid ingress and contact with biological structures, which is further complicated by the need to maintain flexibility in the neural interface 100. Accordingly, specific materials and techniques can be used to hermetically seal the neural interfaces 100 described herein to account for these and other factors.

Fully implanted, subgaleal neural interfaces 100 generally have a desired lifetime of at least five years because they require surgical intervention in order to implant and remove. Because the neural interface 100 resides in contact with the brain for an extended period of time, it can be desirable for the various components of the neural interface 100 (e.g., the electronics) to be hermetically or substantially hermetically encapsulated or sealed for a number of reasons. For example, the components of the neural interface 100 can be constructed from materials (e.g., particular polymers or silicon-based substrates) that are interactive with bodily fluids (e.g., cerebrospinal fluid (CSF)). If exposed to bodily fluids, such materials can dissolve into the subjects' bodies or produce harmful byproducts. For example, silicon is biologically interactive and thus, if exposed to the subject's cortical surface, can dissolve into the subject's body. Additionally, some metals can corrode, delaminate, and leak into the patient's body. Therefore, it can be undesirable to use such materials on components of neural interfaces 100 that can contact bodily fluids and/or materials. Accordingly, disclosed herein are various embodiments of hermetically sealed neural interfaces 100 and techniques for hermetically sealing the same. In some embodiments of the neural interfaces 100 described herein, the neural interface 100 as a whole or individual components thereof can be hermetically sealed with biocompatible and biostable materials. For example, the electrode array (or a portion thereof), the ASIC(s), the passive layer, the lead wire, and a variety of other components of the neural interface 100 or interfaces therebetween can be hermetically sealed with biocompatible and biostable interactive materials.

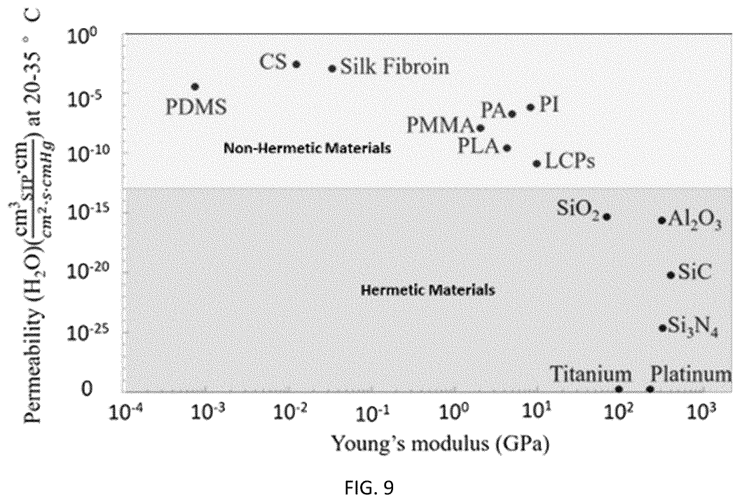

Embodiments of the neural interfaces 100 described herein can include a variety of different materials for hermetically sealed or encapsulating the neural interfaces 100 or components thereof. The materials can include, for example, glass, ceramic, plastics, or metals that are stable in the body (i.e., are substantially nonreactive with biological fluids and biological materials). Certain plastics, for example, have a low water vapor permeability that can be more suited to serve as an encapsulation layer. A low water vapor permeability in materials can be desirable as those materials reduce flow of vapor through a barrier layer. The lower the water vapor permeability, the less water vapor flows through the device, which contributes to the hermeticity of the neural interface 100 encapsulation. For example, polyimide, parylene, or liquid crystal polymer can be used for encapsulating components of the neural interface 100. As another example, FIG. 9 depicts multiple materials that have the requisite physical properties (when thin enough, according to by the Young's modulus) and impermeability to be utilized in connection with various embodiments of the neural interfaces 100 described herein, including SiO₂, Al₂O₃, SiC, Si₃N₄, Ti, and Pt. Various embodiments of the neural interfaces 100 can use one or more of the depicted materials for hermetically sealing components and/or interfaces between components.

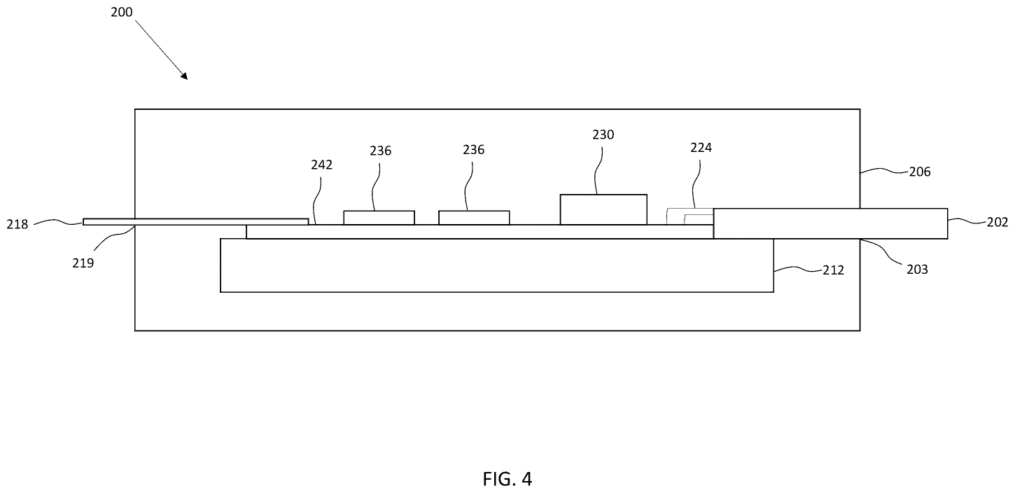

The neural interfaces 100 described herein can have multiple components or interfaces between different components that can be hermetically sealed. In some embodiments, different types of hermetic encapsulations can be used to seal different components or interfaces between particular combinations of components. As shown in FIGS. 1-6 and described above, the neural interfaces 100 can include, for example, an electrode array that is implanted to lay against the cortical surface, electronics (e.g., for processing electrocortical signals), the substrate on which the electronics and other components are disposed, and a transcutaneous lead wire that is used to connect the implanted components to the external components (e.g., a power supply or external transceiver). It can be beneficial to hermetically seal these components and/or the interfaces between these components. In some embodiments, it can be beneficial to seal the aforementioned components and/or the interfaces using materials or techniques that correspond to the structural and material properties of these components and/or interfaces. In one embodiment, the hermetically sealed neural interfaces 100 described herein can include one or more encapsulation layers that are designed to hermetically seal components of the neural interface 100 from biological environments (e.g., the human body). In one embodiment, the neural interface 100 can include an encapsulation layer that is configured to hermetically seal the substrate-array interface, the substrate-electronics interface, and the substrate-lead interface, such as is shown in FIG. 4. In other embodiments, the neural interface 100 can include encapsulation layers for different or additional component, in addition to or in lieu of the aforementioned encapsulation layer. In some embodiments, the encapsulation layers can provide additional functionality beyond hermetically sealing the neural interface 100. For example, an outer encapsulation can mechanically stabilize the neural interface 100.

Various illustrative embodiments of different assemblies and techniques for hermetically sealing neural interfaces 100 are shown in FIGS. 4-6 and described below. It should be noted that the different assemblies and techniques described herein can further be used in combination with each other. The present disclosure is intended to encompass all such modifications and combinations. In particular, FIGS. 4-6 depict various embodiments of an illustrative subgaleal portion 200 portion of a neural interface 100 that are hermetically sealed using different assemblies and techniques. The neural interface 100 includes a substrate 212 to which various other components of the subgaleal portion 200 are affixed, including a redistribution layer (RDL) 242 to which various electronics are connected, including the electrode array 218, one or more application-specific integrated circuits (ASICs) 236, and passives 232. In some embodiments, the substrate 212 can be flexible. In particular, the substrate 212 can be fabricated from flexible materials. The RDL 242 is a partially insulated, patterned metal (e.g., copper) layer that makes I/O pads available to other locations along its length. The subgaleal portion 200 further includes a lead wire 202 that extends transcutaneously from the implanted subgaleal portion 202 to external electronics. The lead wire 202 can be connected to the RDL layer 242 via wire bonds 224. In various embodiments, the subgaleal portion 200 can be dimensioned to fit within the subdural space 204. For example, the subgaleal portion 200 can have a height between about 2 mm and about 10 mm. In one illustrative embodiment, the subgaleal portion 200 can have a height of about 5.1 mm.

In one embodiment shown in FIG. 4, the subgaleal portion 200 portion of the neural interface 110 includes an outer encapsulation layer 206 that encompasses the neural interface electronics subassembly (e.g., the ASICs 236 and the passives 230), the interface between the electrode array 218 and the substrate 212, and the interface between the lead wire 202 and the flexible substrate 212. The encapsulation layer 206 can further include a feedthrough 219 that the distal or cortical surface-contacting portion of the electrode array 218 extends through and a feedthrough 203 that the transcutaneous lead wire 202 extends through. The feedthroughs 203, 219 can be formed via, for example, multilayer encapsulation by hermetic barriers.

In some embodiments, the encapsulation layer 206 can be molded or cast from thermoplastic or thermoset resins. For example, the encapsulation layer 206 can include epoxy resins, silicone or parylene resin coatings, thermoplastics (e.g., polyether ether ketone (PEEK) or liquid crystal polymer (LCP)), fluoro-or chloropolymers, and/or fluoro-or chloroelastomers. In some embodiments, encapsulation layer 206 can include various thin film coatings, such as parylene or various ceramics (e.g., silicon nitride, silicon oxynitride, silicon carbide, aluminum oxide, or transition metal oxides). Such thin film coatings can be deposited using a variety of different techniques, including physical vapor deposition, chemical vapor deposition, sputtering, or atomic layer deposition. In some embodiments, the encapsulation layer 206 can include a single layer or multiple layers of the same or different materials. For example, the encapsulation layer 206 can include a multilayer stack of materials, wherein one layer can function as the chemical interfaces for the underlying component materials and a second layer can serve as a barrier layer selected for its mechanical and/or hermetic properties. In one illustrative embodiment, the encapsulation layer 206 can include parylene and silicone epoxy.

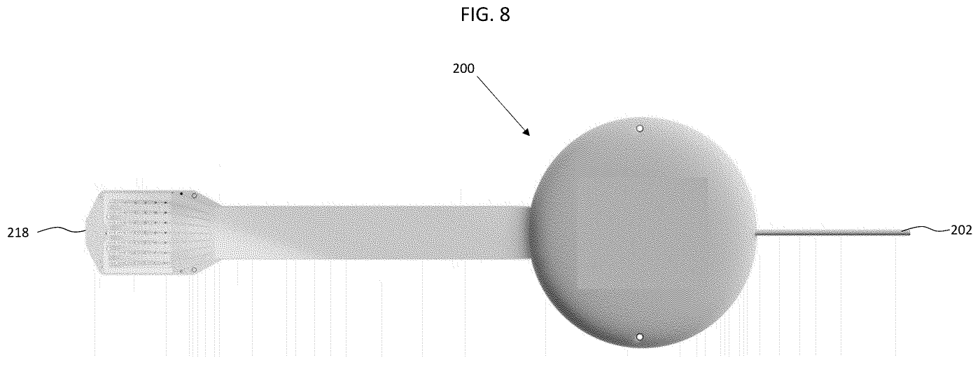

As discussed above, it can be desirable for the feedthroughs 203, 219 to be further sealed in order to ensure that the subgaleal portion 200 is fully hermetically sealed. In these embodiments, the feedthroughs 203, 219 can be formed via, for example, laser drilling through a glass or ceramic substrate such as silicon, aluminum, or zirconium oxide. Different laser drilling techniques can be used to form the feedthroughs 203, 219, including, for example, direct drilling or laser induced deep etching (LIDE). In one embodiment, the feedthroughs 203, 219 can be sealed using electroplating techniques (e.g., using one or more of the hermetic materials listed in FIG. 8) and insertion of an insulating core fill. For example, the feedthroughs 203, 219 can be sealed using seed deposition, followed by inserting plates along the sidewalls of the feedthroughs 203, 219. In another embodiment, the feedthroughs 203, 219 can be sealed using a glass-frit and/or metal-based paste to fill the feedthroughs 203, 219 and sintering. In either of these embodiments, the feedthroughs 203, 219 are further be capped by either a direct surface finish (e.g., electroless nickel immersion gold, particularly if copper is present in the fill/plate) or by a thin film metal to provide an additional seal. Such caps can additionally provide a more appropriate surface to which electrical connections can be bonded.

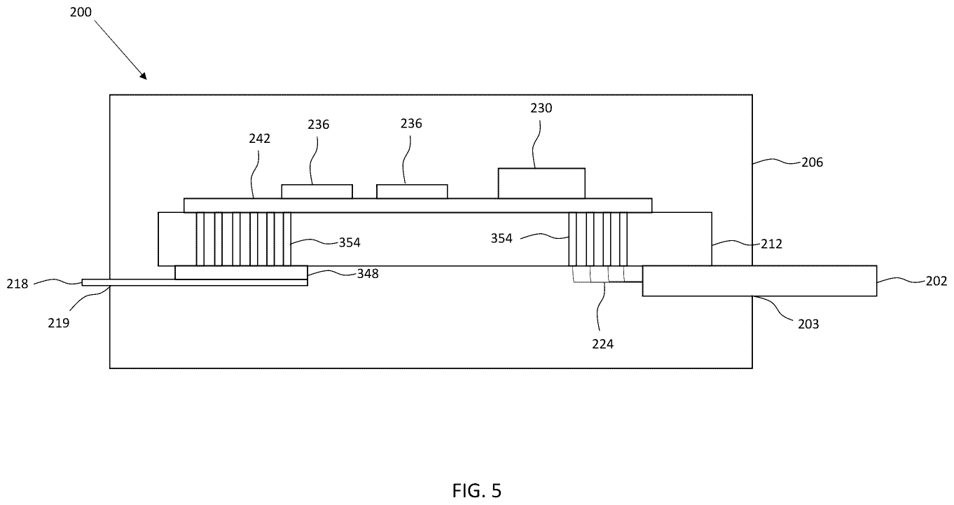

In another embodiment of the subgaleal portion 200 shown in FIG. 5, the electrode array 218 and the lead wire 202 can be bonded to the opposite surface of the substrate 212 from the electronic components (e.g., the RDL layer 242, ASICs 236, and passives 230). In this embodiment, the electrode array 218 and the lead wire 202 can be electrically coupled to the RDL layer 242 by conductive vias 354 extending through the substrate 212. Further, the electrode array 218 can be electrically coupled to the vias 354 by a secondary RDL layer 348. As with the embodiment shown in FIG. 4, the subgaleal portion 200 can include outer encapsulation layer 206 that encompasses the neural interface electronics, the interface between the electrode array 218 and the substrate 212, and the interface between the lead wire 202 and the substrate 212. Further, the encapsulation layer 206 can be constructed from the same materials discussed above and the feedthroughs 203, 219 can be sealed using the same materials and techniques discussed above.

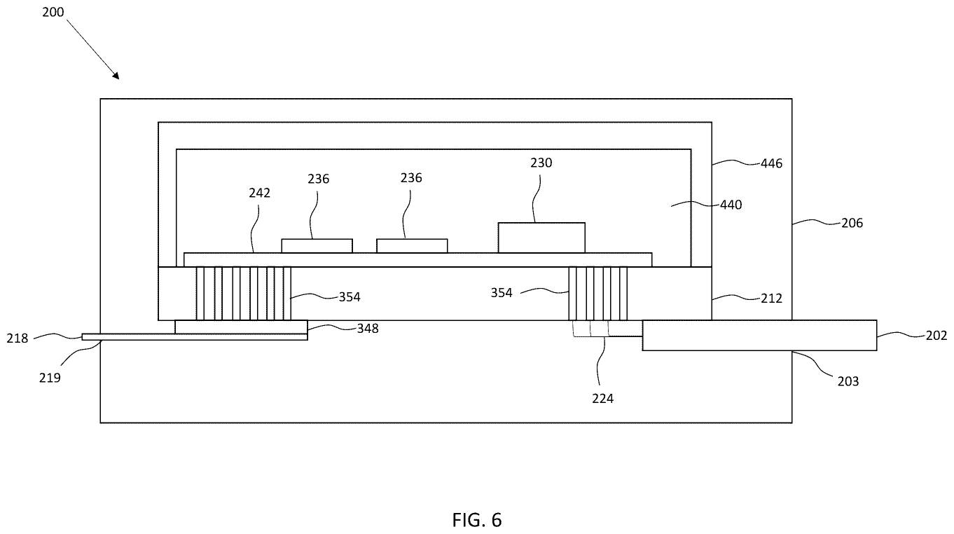

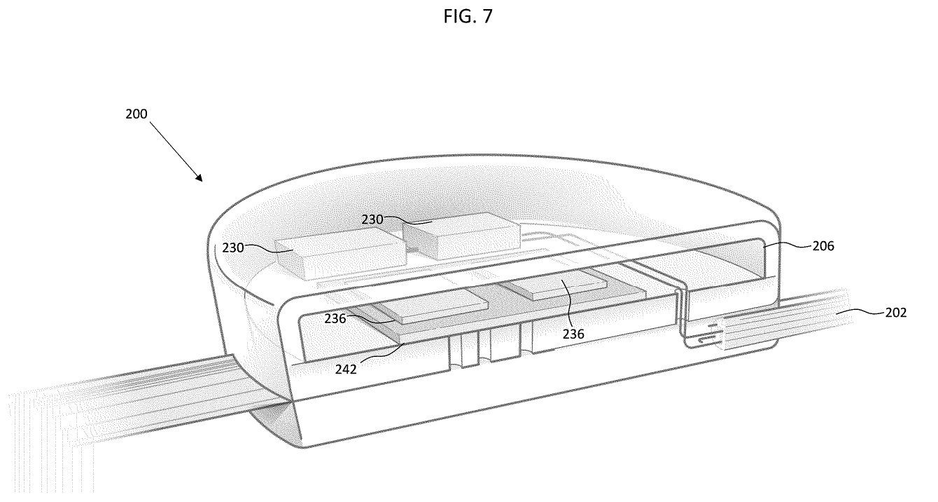

In another embodiment shown in FIG. 6 and FIG. 7, the subgaleal portion 200 of the electrode array 110 can further include multiple encapsulation components. FIG. 6 shows a sectional view of the subgaleal portion 200 and FIG. 7 shows a partially transparent, perspective sectional view where the routing and feedthroughs are depicted as cutaways with material removed from the vias for visibility of the signal path. In particular, the subgaleal portion 200 can include an outer encapsulation layer 206 (as described above with respect to the embodiments shown in FIGS. 4 and 5) and an internal encapsulation assembly 446. In one embodiment, the internal encapsulation assembly 446 can include a glass, ceramic, or biocompatible metal (e.g., titanium) lid formed over the electronic components of the subgaleal portion. The glass lid can form a sealed space 440 encompassing the ASICs 236 and the passives 230, for example. The encapsulation layer 206 can then be formed over the internal encapsulation assembly 446 to further seal the aforementioned electronics, as well as the interface between the electrode array 218 and the substrate 212 and the interface between the lead wire 202 and the substrate 212. In this embodiment, the outer encapsulation layer 206 can serve as the primary outer barrier for the internal electronics and the internal encapsulation assembly 446 can serve as a secondary barrier.

In some embodiments, an internal encapsulation assembly 446 can be configured to be sealed with an inert environment. In some embodiment, the encapsulation assembly 446 can have a thickness from about 300 μm to about 500 μm. The cavity sealed space 440 can have a height of approximately 800 μm. The packaging system 400 can be considered fully hermetic as a glass, ceramic, or metal-ceramic package for the electronics, such as the ASIC 246, underneath the lid 446. The internal encapsulation assembly 446 can be attached to the substrate 212 via laser welding, glass frit scaling, (glass-metal)-(metal-glass) sealing, reactive metal welding, or other such techniques prior to the application of the outer encapsulation layer 206.

In some embodiments, the internal electronic components (described above) of the subgaleal portion 200 can be bonded using conventional solders (e.g., SAC305) to the RDL layer 242 to form the subgaleal implant electronics. The electronic components can be underfilled and overmolded by epoxies, silicones, or other resins, either with or without thermal components to direct heat away from the brain.

In some embodiments, the encapsulation layer 206 can be configured to provide additional mechanical stability, in addition to hermetically sealing the subgaleal portion 200 of the neural interface 110. Accordingly, the material(s) of the encapsulation layer 206 can be selected to provide appropriate chemical bonding characteristics to the underlying components, while still providing the desired mechanical characteristics (e.g., flexibility and/or rigidity) as needed to support the substrate 212 and allow for the subgaleal portion 200 to be inserted using the minimally invasive techniques described above.

In some embodiments, the external encapsulation layer 206 can be composed of multiple different types of materials. The chosen material of the external encapsulation layer 206 can be determined based on desired adhesion and intrinsic barrier properties, such as water vapor permeability or biostability. For example, the materials selected for the external encapsulation layer 206 can provide low water vapor permeability. If multiple layers are desired for the external encapsulation layer 206, the multiple layers can be stacked to provide protection against water vapor ingress and corrosion by body fluids, as described above.

In some embodiments, the external encapsulation layer 206 can include multilayer stacks that can be selectively masked using photosensitive or non-photosensitive polymer masking layers (e.g., a photoresist, latex, or other dissolvable or peelable plastics). The masking coats a portion of the electrode array 218 that extends from the subgaleal portion 200, without fully coating the electrode surfaces of the electrode array 218.

In one embodiment, the encapsulation layer 206 can include three or more materials. For example, the encapsulation layer 206 can include a multilayer stack of silicone-parylene-silicone. The first layer of silicone can be dip coated using chemical primers and chemical treatments (e.g., plasma treatment using O₂, N₂, Ar, CFₓ) to establish maximum chemical interaction and adhesion with the substrate 212, electrode array 218, and/or lead wire 202. The parylene layer can serve as a conformal barrier coating across the subgaleal portion 200. The final silicone layer (which can include a thermoset or thermoplastic layer, such as LCP, fluoroelastomers, chloroelastomers, PEEK, or epoxy) can further be configured to mechanically protect the underlying parylene layer.

In another embodiment, the subgaleal portion can include a resin layer, followed by a barrier, and an encapsulation layer 206 as described above. The resin layer can include various primers and surface treatments compatible with the underlying materials and/or components of the subgaleal portion 200. The barrier of the resin-barrier encapsulation layer 206 can include low vapor permeability materials, such as parylene, ceramics (e.g., silicon nitride, silicon oxynitride, silicon carbide, or aluminum oxide), transition metal oxides, or any combination thereof. In some embodiments, the transition metal oxides can be deposited by plasma enhanced chemical vapor deposition (CVD), chemical reactions, atomic layer deposition (e.g., plasma-enhanced), and sputtering (e.g., reactive sputtering). The encapsulation layer 206 can serve as the outer mechanical or barrier layer and can include thermoplastics and thermoset polymers (e.g., PEEK, LCP, epoxy, or silicones). Further, the encapsulation layer 206 can end before or beyond the extent of resin layer and/or the barrier on the lead wire 202. That is, the resin layer, barrier layer, or the encapsulation layer 206 can cover the lead wire 202 enough such that maximum adhesion to the polyimide outer surface of the lead wire 202 occurs. Controlling the extent to which the lead wire 202 is covered by the resin layer, barrier layer, and/or encapsulation layer 206 can allow one to customize the desired adhesion and/or mechanical encapsulation of the other layers in the stack.

In some embodiments, the external encapsulation layer 206 can have additional mechanical features to aid in surgery, handling, or interface with other systems or devices. These features can include, but are not limited to, loops, tabs, mounting holes, or other features that match anatomical or surgical shapes such as skull curvature, bone recesses, and re-placed skin. Further, some embodiments can include additional material (e.g., reinforcing metal or polymer) embedded in or on the external encapsulation layer 206 for use with bone screws, sutures, and other surgical features. In various embodiments, the encapsulation layer 206 can be formed into a variety of different shapes, which can aid in the direction, strain relief, and handling of the electrode array 218. Further, the encapsulation layer 206 can further be shaped to be compatible with tunneling devices (which can be used in connection the lead wire 202, for example). Still further, the external encapsulation layer 206 can include mounting features for endoscopes or other surgical tools. These additional features and/or materials can be embedded in or affixed to the encapsulation layer 206.

Referring now to FIG. 8, there is shown a fully encapsulated representation of the embodiment of the embodiment of the subgaleal portion 200 shown in FIG. 6 and FIG. 7. As can be seen, the techniques and components described herein can be used to form fully sealed housings for sealing the electronics and other components of the subgaleal portion 200 of a neural implant from biological environments.

While the foregoing is directed to embodiments described herein, other and further embodiments can be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and may be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A neural interface for subdural implantation, the neural interface comprising:
a substrate;
an electronics subassembly disposed on the substrate;
an electrode array coupled to the substrate defining a first interface, wherein the electrode array comprises a plurality of electrodes disposed at a distal end thereof;
a lead wire coupled to the substrate defining a second interface; and
an encapsulation layer covering the electronics subassembly, the first interface, and the second interface, wherein the encapsulation layer comprises a low vapor permeability material, and wherein the encapsulation layer hermetically seals the electronics subassembly, the first interface, and the second interface from a biological environment.

2. The neural interface of claim 1, wherein the substrate is flexible.

3. The neural interface of claim 1, wherein the electronics subassembly comprise an application-specific integrated circuit.

4. The neural interface of claim 1, wherein the encapsulation layer comprises a multilayer stack of materials.

5. The neural interface of claim 1, wherein the low vapor permeability material comprises a glass, a plastic, a ceramic, or a combination thereof.

6. The neural interface of claim 1, wherein the electronics subassembly comprises at least one of a redistribution layer, a passive layer, or an application-specific integrated circuit.

7. The neural interface of claim 1, wherein the encapsulation layer comprises a thin film encapsulation layer.

8. The neural interface of claim 1, wherein the encapsulation layer comprises at least one mechanical feature configured to interface with external systems.

9. The neural interface of claim 8, wherein the at least one mechanical feature is at least one of a loop, mounting hole, a matching shape, or a mount.

10. The neural interface of claim 1, further comprising an internal encapsulation assembly configured to cover the electronics subassembly, wherein the encapsulation layer covers the internal encapsulation assembly.

11. The neural interface of claim 10, wherein a material of the internal encapsulation assembly comprises at least one of glass, ceramic, or biocompatible metal.

12. The neural interface of claim 10, wherein the internal encapsulation assembly defines an inert sealed interior cavity.

13. The neural interface of claim 1, wherein the electrode array comprises 1,000 or more electrodes.

14. A method of fabricating a neural interface for subdural implantation, the method comprising:
attaching an electrode array to a first portion of a substrate defining a first interface, wherein the electrode array comprises a plurality of electrodes;
attaching a lead wire to a second portion of the substrate defining a second interface;
attaching an electronics subassembly to the substrate; and
forming an encapsulation layer covering the first interface, the second interface, and the electronics subassembly, wherein the encapsulation layer comprises a low vapor permeability material, wherein the encapsulation layer hermetically seals the electronics subassembly, the first interface, and the second interface from a biological environment.

15. The method of claim 14, wherein the encapsulation layer is formed by welding or sealing the encapsulation layer to the substrate.

16. The method of claim 14, wherein the substrate is flexible.

17. The method of claim 14, wherein the electronics subassembly comprise an application-specific integrated circuit.

18. The method of claim 14, wherein the encapsulation layer comprises a multilayer stack of materials.

19. The method of claim 14, wherein the low vapor permeability material comprises a glass, a plastic, a ceramic, or a combination thereof.

20. The method of claim 14, wherein the electronics subassembly comprises at least one of a redistribution layer, a passive layer, or an application-specific integrated circuit.

21. The method of claim 14, wherein the encapsulation layer comprises a thin film encapsulation layer.

22. The method of claim 14, wherein the encapsulation layer comprises at least one mechanical feature configured to interface with external systems.

23. The method of claim 22, wherein the at least one mechanical feature is at least one of a loop, mounting hole, a matching shape, or a mount.

24. The method of claim 14, further comprising an internal encapsulation assembly configured to cover the electronics subassembly, wherein the encapsulation layer covers the internal encapsulation assembly.

25. The method of claim 24, wherein a material of the internal encapsulation assembly comprises at least one of glass, ceramic, or biocompatible metal.

26. The method of claim 24, wherein the internal encapsulation assembly defines an inert sealed interior cavity.

27. The method of claim 14, wherein the electrode array comprises 1,000 or more electrodes.
